(19) United States
(12) Patent Application Publication
(10) Pub. No.: US 2022/0087911 A1
(43) Pub. Date: Mar. 24, 2022

(54) DENTAL FLOSS FOR PREVENTING OR TREATING DENTAL CARIES AND PERIODONTAL DISEASE

(71) Applicant: UNIVERSIDAD DE LOS ANDES, Las Condes (CL)

(72) Inventor: Ziyad Samir HAIDAR, Las Condes (CL)

(21) Appl. No.: 17/425,253

(22) PCT Filed: Jan. 22, 2020
(86) PCT No.: PCT/IB2020/050497

§ 371 Date: Jul. 22, 2021

(30) Foreign Application Priority Data
Jan. 22, 2019 (EP) 19153063.3

(51) Int. Cl. A61K 8/19; A61C 15/04; A61K 8/02; A61Q 11/00

(52) U.S. Cl. CPC A61K 8/19; A61Q 11/00; A61K 8/0204; A61C 15/041

(57) ABSTRACT

The present invention refers to the oral and dental health care field since it refers to a modified and improved dental floss material aimed at preventing the onset and progression of dental caries and periodontal disease.

### FIELD OF THE INVENTION

The present invention refers to the medical field. More specifically, the present invention refers to the odontology (oro-dental) field since it is focused on an improved/modified dental floss aimed at preventing or treating dental caries and periodontal disease. The dental floss of the invention can be used as an adjunct bio-maintenance tool to clinical intervention, for example following professional scaling and root planning by a specialist dentist to maintain efficacy of therapy.

### STATE OF THE ART

Under natural settings, oral bacteria tend to accumulate and assemble in complex poly-microbial communities (biofilm) that attach over both, biotic and abiotic intraoral surfaces (gingiva and teeth respectively). As the surfaces become colonized, the biofilm matures by (a) expanding (becoming larger) and (b) adopting a unique architecture, which allows it not only to exhibit biological and functional heterogeneity that enhances its intraoral survival (i.e. separate regions of fast- and slow-growing bacteria that functions as a “genomic reservoir” of specific bacterial strains), but also to produce/express different acids and proteins (including pro-inflammatory cytokines) which set the onset and progression of gingival inflammation, periodontal disease and dental decay (dental caries).

Oral cleanliness and biofilm (dental plaque) control/removal is considered to be essential for preserving oral health and preventing progress of dental caries, gingival and periodontal disease. If untreated, both conditions contribute to tooth decay, periodontal tissue destruction and eventual tooth loss, diminishing not only the quality of life of patients but also affecting their systemic, nutritional and psychological health/status (i.e. self-confidence, social interaction, personal satisfaction with aesthetics, etc.). Oral cleanliness by traditional tooth brushing removes some of the dental plaque on the surface of teeth and gum line (up to 60% of the overall oral dental plaque). Nonetheless, it does not reach the area in-between teeth (inter-proximal or inter-dental area) where gum disease and caries are most common.

The interproximal area also presents unique histological conditions which facilitate the development of caries and periodontal disease, such as (1) difficult access and a (2) particularly thin gingival epithelium (which allows the rapid invasion of bacteria into the gingival tissues), reason for which the regular use of specially designed instruments/tools for accessing and cleaning the area are often encouraged in addition to usual self-performed tooth-brushing. In fact, several health agencies including the American Dental Association (ADA), The General Surgeon, The Center for Disease Control (CDC) and The American Academy of Periodontology (AAP), continue to recommend cleaning “at least once a day” with an inter-dental gadget in order to maintain a healthy smile (in spite of recent controversies published by the media in the last months). At the present, many different products have been developed and commercialized to achieve this goal, including: dental flosses, wood tooth-picks, inter-proximal brushes and oral irrigators. While current scientific evidence may not answer “which inter-proximal gadget is the best” (due to limited high-quality evidence), clinicians agree on that not all interdental devices suit all patients or all types of dentition. More importantly, patient's motivation is a crucial factor when recommending an interdental cleaning method because the regular use and compliance is essential to obtain good results.

The concept of “flossing” for cleaning the interproximal area appears to have been first introduced by Parmly and col. in 1819. Since then, dental floss has been considered a simple, systematic and patient-friendly method for inter-dental plaque removal. As such, it is the most frequently recommended interproximal cleaning gadget in the market though to several advantageous characteristics such as: (1) it may be performed in nearly all circumstances by most patients, (2) accesses not only the interproximal area but also the subgingival space (a distinctive quality vs. other devises such as interproximal brushes), (3) is the most cost-friendly or inexpensive among interdental instruments and (4) is widely available and advertised in the market (most patients are familiar with it and know how to use it). Nonetheless, compliance with regular flossing continues to be low due to patient's lack of (a) motivation and (b) ability for its correct use. Dental floss is the most frequently recommended and advertised inter-dental cleaning tool in the market making up to 16% of the overall “over-the-counter” dental product market. As such, most patients are familiarized with it. According to recent data from Euromonitor International Market Research, dental floss sales in the United States increased up to 12% in the past decade with Americans spending an estimated $448 million dollars on dental floss just in 2015.

Knowing that the dental floss has not been significantly improved for decades, and that it is an important tool for preventing dental caries and periodontal disease, the present invention refers for the first time to a modified or a bio-improved dental floss for the localized delivery and controlled release of anti-bacterial, antifungal or immunostimulating molecules directly into the interproximal and supra/subgingival areas of the tooth. Regular use of the floss developed in the present invention should significantly reduce the incidence of gum disease and dental caries while providing all benefits usually associated with mechanical flossing.

### DESCRIPTION OF THE INVENTION

### Brief Description of the Invention

The present invention refers to an improved dental floss (dental floss of the invention) focused on solving two main problems: (i) Improving the hygiene of the interproximal supra and subgingival areas via mechanical removal/elimination of dental plaque and food particles located in the aforementioned space, and (ii) Prevention of dental caries and periodontal diseases via direct, localized and “controlled” release-delivery of mouth-dissolving, biodegradable, tooth-muco-adhesive compositions comprising antibacterial, antifungal and/or immunostimulating active ingredients into the interproximal area.

The dental floss of the invention is designed as a bio-active dental floss tool due to incorporating into the floss itself anti-bacterial compounds which benefits or relies on its localized delivery to the site “pocket” or accumulation within the “gingival crevice” to have a significant effect on preventing disease progression. So, the dental floss of the invention can be defined as an adjunct tool to professional dental treatment/cleaning where the dentist would recommend its use after providing the therapy to maintain the effect of the clinical intervention. Thus, the dental floss of the invention can be used by consumers to replace their current traditional non-bioactive flosses tools in order to have a superior cleaning and long-lasting effect via diseases onset and/or progress prevention.

The dental floss of the invention is coated with a very specific composition, which comprises at least one natural occurring polymer (which is biodegradable, mouth-dissolving and tooth-muco-adhesive) and, preferably, also active ingredients (such as antibacterial, antifungal and immunomodulatory agents), aimed at preventing caries and/or periodontal disease. In a preferred embodiment of the invention, the composition used for coating the dental floss of the invention is specially designed for the localized and controlled release delivery of said active ingredients directly into the interproximal and subgingival areas.

Thus, the present invention refers to a method for modifying (briefly, via polymer coating and nanoparticle loading or incorporation within) a regular or traditional silk or nylon flossing materials (thread) to obtain a bioactive dental floss comprising the steps: a) Preparing a composition comprising at least one natural occurring polymer and at least one antibacterial, antifungal and/or immunostimulating active ingredient and b) step-wise adsorption of different polymer coating assembled layer-by-layer onto the floss thereby creating multi-compartments around it able to be loaded with therapeutic or preventive active ingredients, and c) drying the composition prepared according to the step a) on the dental floss. In a preferred embodiment on the invention the step b) is preferably carried out by dipping the dental floss into the composition prepared according to the step a). In a preferred embodiment of the invention, the natural occurring polymer is selected from the list comprising: chitosan, gelatine, alginate, cellulose, hyaluronic acid, albumin, or any salts derived thereof. In a preferred embodiment of the invention, the antibacterial, antifungal and/or immunostimulating active ingredient is selected from the list comprising: Copper, silver, lithium, chlorhexidine, fluoride or any salt derived thereof.

In a preferred embodiment of the invention, the composition according to the step a) comprises nanocapsules having a polymeric membrane made of at least one natural occurring polymer and at least one active principle encapsulated inside the polymeric membrane. In a preferred embodiment of the invention, the composition according to the step a) comprises metal nanoparticles coated with a polymeric membrane made of at least one natural occurring polymer. In a preferred embodiment of the invention, the natural occurring polymer is selected from the list comprising: chitosan, gelatine, alginate, cellulose, hyaluronic acid, albumin, or any salts derived thereof; and the active ingredient is selected from the list comprising: Copper, silver, lithium, chlorhexidine, fluoride or any salt thereof. In a preferred embodiment of the invention, the natural occurring polymer is selected from the list comprising: chitosan, gelatine, alginate, cellulose, hyaluronic acid, albumin, or any salts derived thereof; and the metal is an active ingredient selected from the list comprising: Copper, silver, lithium or any salt thereof. In a preferred embodiment of the invention, the polymeric membrane is prepared by the layer-by-layer self-assembly of the finely-tuned polymer blends based on electrostatic interactions, with controlled physic-chemical-mechanical and pharmacokinetic properties.

It is important to note that by means of the method carried out in the present invention, strong covalent interactions are generated between the natural occurring polymer/s and the dental floss, thus giving rise to a stable dental floss with the capability to be loaded with bio-agents and be used for preventing caries and periodontal disease or maintaining professional/clinical therapy to the pre-mentioned diseases/conditions.

The present invention also refers to a dental floss coated with a composition comprising at least one natural occurring polymer and at least one antibacterial, antifungal and/or immunostimulating active ingredient. In a preferred embodiment of the invention, the natural occurring polymer is selected from the list comprising: chitosan, gelatine, alginate, cellulose, hyaluronic acid, albumin, or any salts derived thereof. In a preferred embodiment of the invention, the antibacterial, antifungal and/or immunostimulating active ingredient is selected from the list comprising: Copper, silver, lithium, chlorhexidine, fluoride or any salt derived thereof. In a preferred embodiment of the invention, the composition comprises nanocapsules having a polymeric membrane made of at least one natural occurring polymer and at least one active principle encapsulated inside the polymeric membrane. In a preferred embodiment of the invention, the composition comprises metal nanoparticles coated with a polymeric membrane made of at least one natural occurring polymer. In a preferred embodiment of the invention, the natural occurring polymer is selected from the list comprising: chitosan, gelatine, alginate, cellulose, hyaluronic acid, albumin, or any salts derived thereof; and the active ingredient is selected from the list comprising: Copper, silver, lithium, chlorhexidine, fluoride or any salt thereof. In a preferred embodiment of the invention, the natural occurring polymer is selected from the list comprising: chitosan, gelatine, alginate, cellulose, hyaluronic acid, albumin, or any salts derived thereof; and the metal is an active ingredient selected from the list comprising: Copper, silver, lithium or any salt thereof.

The invention also refers to a method for preventing onset and progress of dental caries and/or periodontal disease which comprises the use of a dental floss defined above.

It is important to note that, such as it is shown in Example 2, copper nanoparticles assayed in the invention are especially suitable for the inhibition of the following bacterial strains: Streptococcus mutans serotype C, Streptococcus mutans serotype K, Streptococcus mutans serotype E, Streptococcus mutans serotype C (ATCC 25175), Staphylococcus epidermidis. In fact, Staphylococcus epidermidis is one of most prevalent in dental caries or dental pulp which has the capability of horizontal genetic transfer between different bacterial species in the oropharynx, suggesting that it may evolve with the dissemination of resistant determinants [Devang Divakar et al., 2017. High proportions of Staphylococcus epidermidis in dental caries harbor multiple classes of antibiotics resistance, significantly increase inflammatory interleukins in dental pulps. Microb Pathog. 2017 August; 109:29-34. doi: 10.1016/j.micpath.2017.05.017. Epub 2017 May 12]. Moreover, Streptococcus mutans is commonly found in the human oral cavity and is a significant contributor to tooth decay [Ryan K J, Ray C G, eds. (2004). Sherris Medical Microbiology (4th ed.). McGraw Hill. ISBN 0-8385-8529-9] [Loesche W J (1996). “Ch. 99: Microbiology of Dental Decay and Periodontal Disease”. In Baron S; et al. Baron's Medical Microbiology (4th ed.). University of Texas Medical Branch. ISBN 0-9631172-1-1. PMID 21413316].

So, the first embodiment of the present invention refers to a dental floss coated with a composition comprising metal nanoparticles which comprises at least one antibacterial, antifungal and/or immunostimulating active ingredient. In a preferred embodiment, the metal nanoparticles have a polymeric membrane made of at least one natural occurring polymer. In a preferred embodiment, the nanoparticle is a copper, silver or lithium nanoparticle. In a preferred embodiment, the natural occurring polymer is selected from the list comprising: chitosan, gelatine, alginate, cellulose, hyaluronic acid, albumin, or any salts derived thereof. In a preferred embodiment, the dental floss is coated with copper nanoparticles having a polymeric membrane comprising chitosan.

The second embodiment of the present invention refers to the use of metal-based nanoparticles comprising at least one antibacterial, antifungal and/or immunostimulating active ingredient for coating a natural polymer-modified dental floss. In a preferred embodiment, the metal nanoparticles have a polymeric membrane made of at least one natural occurring polymer (i.e. core-shell nanocapsules). In a preferred embodiment, the nanoparticle is a copper, silver or lithium nanoparticle. In a preferred embodiment, the natural occurring polymer is selected from the list comprising: chitosan, gelatine, alginate, cellulose, hyaluronic acid, albumin, or any salts derived thereof. In a preferred embodiment, the nanoparticles are copper nanoparticles having a polymeric membrane comprising chitosan. The benefit or purpose of modifying metal-based nanoparticles with natural polymer(s) is to maintain stability, control/modulate dose-response and control/modulate release kinetics.

The third embodiment of the present invention refers to metal nanoparticles comprising at least one antibacterial, antifungal and/or immunostimulating active ingredient for use in preventing caries and/or periodontal disease. In a preferred embodiment, the nanoparticles have a polymeric membrane made of at least one natural occurring polymer. In a preferred embodiment, the nanoparticle is a copper, silver or lithium nanoparticle. In a preferred embodiment, the nanoparticles are copper nanoparticles having a polymeric membrane comprising chitosan. In a preferred embodiment, the prevention of caries and/or periodontal disease is carried out by inhibiting the bacteria strains Streptococcus mutans serotype C, Streptococcus mutans serotype K, Streptococcus mutans serotype E, Streptococcus mutans serotype C (ATCC 25175) or Staphylococcus epidermidis. In a preferred embodiment, the metal nanoparticle is incorporated/loaded within the dental floss framework, and in case of a single-filament (mono-filament) dental floss, then around it.

The fourth embodiment of the present invention refers to a method for obtaining a bioactive dental floss as defined above comprising the steps: a) Preparing a composition which comprises metal nanoparticles comprising at least one antibacterial, antifungal and/or immunostimulating active ingredient, b) Dipping the dental floss into the composition prepared according to the step a), and c) Drying the composition prepared according to the step a) on the dental floss. In a preferred embodiment, the metal nanoparticles have a polymeric membrane made of at least one natural occurring polymer. In a preferred embodiment, the nanoparticle is a copper, silver or lithium nanoparticle. In a preferred embodiment, the natural occurring polymer is selected from the list comprising: chitosan, gelatine, alginate, cellulose, hyaluronic acid, albumin, or any salts derived thereof. In a preferred embodiment, the polymeric membrane is prepared by layer-by-layer self-assembly of the polymer blends.

The fifth embodiment of the present invention refers to a method for preventing caries and/or periodontal disease which comprises the use of a dental floss as defined above.

For the purpose of the present invention the following definitions are provided:
The term “metal nanoparticle” refers to particles between 1 and 100 nanometres in size comprising any element classified as metal on the periodic table, for example copper, lithium or silver. In fact, the periodic table comprises a stair-stepped line starting at Boron (B), atomic number 5, and going all the way down to Polonium (Po), atomic number 84. Except for Germanium (Ge) and Antimony (Sb), all the elements to the left of that line can be classified as metals.
The term “bioactive dental floss” refers to a dental floss which has a biological effect on a living organism. In this case, the dental floss is “bioactive” because it is able to inhibit bacteria which are responsible for causing caries and/or periodontal disease.
The term “active ingredient” refers to a substance in the composition that provides a desired effect, particularly being able to prevent caries or periodontal disease.
The term “comprising” it is meant including, but not limited to, whatever follows the word “comprising”. Thus, use of the term “comprising” indicates that the listed elements are required or mandatory, but that other elements are optional and may or may not be present.
By “consisting of” is meant including, and limited to, whatever follows the phrase “consisting of”. Thus, the phrase “consisting of” indicates that the listed elements are required or mandatory, and that no other elements may be present.

### BRIEF DESCRIPTION OF THE FIGURES

FIG. 1. SEM images showing the incorporation of copper nanoparticles (NpCu) (black arrows) on the surface of the dental floss. Morphology and distribution in multifilament dental floss (Photos C and D, the black arrows indicate groups of NpCu). (Photos A and B, untreated dental floss controls).

FIG. 2. Bacterial strains Streptococcus mutans serotype C (A), Streptococcus mutans serotype K (B), Streptococcus mutans serotype E (C), Streptococcus mutans serotype C (ATCC 25175) (D), Staphylococcus epidermidis (E) were cultured in optimal conditions for 24 hours and the formation of halos of inhibition was observed (see black arrows).

FIG. 3. SEM images. Close view of a group of NpCu.

FIGS. 4, 5 and 6. Scanning electron microscopy with different treatments. Longitudinal view. Different magnitudes.

FIG. 7. It shows the release of NPCu from thread samples coated with polymer (s). X axis: % of release. Y axis: Time (minutes).

FIG. 8. Force vs. time graph for resistance measurement (A) Graph with a break point and (B) Graph with several break points. (Arrows: Break points). X axis: time (seconds). Y Axis (Force in grams).

FIG. 9. Minimum Inhibitory Concentration for NPCu in L. Casei. X axis (days).

FIG. 10. Minimum Inhibitory Concentration for NPCu in L. Paracasei. X axis (days).

FIG. 11. Minimum Inhibitory Concentration for NPCu in S. mutans. X axis (days).

FIG. 12. Formation of biofilms in the presence of NPCu in L. Casei.

FIG. 13. Formation of Biofilms in the presence of NPCu in L. Paracasei.

FIG. 14. Formation of biofilms with saliva inoculum from 4 different donors. X axis (donor 1, donor 2, donor 3 and donor 4).

FIG. 15. It shows that the first concentration of NPCu with a significant decrease in biofilm formation is at 2000 ppm.

FIG. 16. Represents the percentage of cell viability in Cal27 cells exposed to different concentrations of NpCu (n=4).

FIG. 17. Represents the percentage of cell viability in MC3T3-E1 cells exposed to different concentrations of NpCu (n=4).

FIG. 18. Represents the percentage of cell viability in MC3T3-E1 cells exposed to different concentrations of NpCu (AlCh) 5 (n=1). X axis (particles/ml).

FIG. 19. Represents the percentage of cell viability in MC3T3-E1 cells exposed to different concentrations of NpCu (AlCh) 5 from powdered NPCu (n=1). X axis (particles/ml).

FIG. 20. Represents the percentage of cell viability in SCC-9 cells exposed to different concentrations of NpCu. (n=4). In SCC-9 cells, cytotoxicity of NPCu from 1000 ppm is observed.

FIG. 21. Represents the percentage of cell viability in SCC-9 cells exposed to different concentrations of NpCu (AlCh) 5 (n=1). X axis (particles/ml),

FIG. 22. Represents the percentage of cell viability in SCC-9 cells exposed to different concentrations of NpCu (AlCh) 5 from powdered NPCu (n=1).

FIG. 23. Represents the percentage of cell viability in HDFn cells exposed to different concentrations of NpCu (n=4).

FIG. 24. Represents the percentage of cell viability in HDFn cells exposed to different concentrations of NpCu (AlCh) 5 (n=1). X axis (particles/m1).

FIG. 25. Represents the percentage of cell viability in HDFn cells exposed to different concentrations of NpCu (AlCh) 5 from powdered NPCu (n=1). X axis (particles/ml).

### DETAILED DESCRIPTION OF THE INVENTION

### Example 1. Material and Methods

### Example 1.1. Preparation and Characterization of the Natural-Polymer Based Nanoformulation

Raw copper nanoparticles (NpCu) (copper, in powered format) were used in the present invention for the development of nanocapsules (as mentioned earlier, suggest distinguishing between using the formulated metal-based nanoparticles and core-shell nanocapsules). Step-wise Layer-by-Layer adsorption (L-b-L Coating) was used for the preparation of the dental floss of the invention. Briefly, fresh 1 mg/mL solutions of all-natural polymeric constituents (i.e. Chitosan, Alginate, Cellulose, Hyaluronic Acid, to mention a few) are prepared in Ultra Pure Water (18.2 MΩcm−1). Chitosan solution, for example, is prepared in 1% (v/v) acetic acid aqueous solution and the final pH adjusted with 1M NaOH to 5.5. Overnight stirring and filtration follows. For the layer-by-layer self-assembly of polymer blends (derived from 32 Full-Factorial analysis), alternating layers of negatively charged polymers and positively charged polymers (volume ratio of 1:2) were incubated at room temperature for 30 minutes under gentle stirring. For the nanoparticles, multi-step filtration will be employed to achieve an average size of 200 nm nanocapsules (monodisperse). Centrifugation at 1500 g for 10 minutes will follow in order to eliminate aggregates that may form upon the mixture of polymeric material in coating and incorporated nanocapsules (washing). Before proceeding to anti-microbial agent loading and/or solvent casting, samples of the multi-layered polymer mixtures/blends are allowed to stand for 3 days, at room temperature, in fridge and freezer, to observe any changes in stability, phase-separation or colour, for the physicochemical, mechanical and rheological characterization.

Regarding the dental floss characterization, briefly average hydrodynamic diameter and zeta potential surface charge of the formulated core-shell nanocapsules (unloaded and loaded) are evaluated via dynamic light scattering at 25° C. with a fixed angle of 90 degrees. Morphology of the full system will be observed using scanning as well as transmission electron microscopy. Loading efficiency and drug release kinetics are quantified by HPLC at different times, in culture medium. Pharmacokinetic modelling (Tri-phasic release kinetics) follows. MTT assay is performed on fibroblasts (HEK-293, ATCC™) and osteoblasts (bone-marrow derived cell-line) to assess cyto-viability.

### Example 1.2. Evaluation of the Anti-Microbial Efficiency Properties of Single and Combined Synergistic Agents Against Cariogenic and Periodontal Disease Pathogens

Anti-microbial effect is determined using the modified agar well-diffusion method, this are carried out with the typically-identified and -recommended representative bacteria for use in (dental) anti-microbial assays. Streptococcus mutans and Streptococcus sanguinis, sub-cultured in 5% blood agar, overnight, for five colonies to be obtained, diluted and incubated under aerobic conditions for 1-2 hours at 37° C. to reach the concentration of 1.5×108 colony-forming units (CFU)/mL (presented as log 10 CFU/mL). Further dilution with a saline solution, to a final concentration of 1.5×106 CFU/mL, will follow. Note: The number of bacterial cells in suspension in all experiments is adjusted. Determination of MIC (minimum inhibitory concentration: lowest concentration of each anti-microbial agent that inhibits the growth of the microorganisms under testing) and MBC (minimum bactericidal concentration: lowest concentration of an anti-microbial agent killing the majority of bacterial inoculums) will be from a known concentration (m/mL) of the 3 anti-microbial agents of choice (Copper, Chlorhexidine and Fluoride), using the liquid micro-dilution method, in our microbiology laboratory. (i) Simulated Oral Media, using artificial saliva, is used for the serial dilution process at pre-determined standardized cut-off points. Diluted micro-organisms (0.5 mL) will be placed in tubes prepared with different concentrations of the antimicrobial agents. Overnight incubation at 37° C. in a closed environment follows. Spectrophotometry (Eppendrof AG, Hamburg, Germany) will be used to measure (turbidity and lack thereof) and determine MIC, after which, an additional sub-culturing and overnight incubation step for a sub-sample is done for MBC to be calculated (numbers of colonies growing from each test tubes are counted and the number of colonies corresponding to a 1000-fold reduction is recorded as the minimum bactericidal concentration). To determine the required time before initiating bactericidal effect, 50 mL of each test specimen will be mixed with 50 mL of the bacterial suspensions (containing 5×103 colonies). Timed culturing and overnight incubation at 37° C., the remaining colonies will be counted. (ii) Human-derived Salivary Microflora, using saliva is collected from subjects attending the Clínica Odontológica (private)-Clínica Universidad de Los Andes (Las Condes, Santiago) and the Clínica Odontológica (public)-Centro de Salud de la Universidad de Los Andes (San Bernardo, Santiago), with no history of antibiotic therapy, use of chemical anti-plaque agents, and oro-dental intervention, prior to six months, of saliva collection for this study. Simply, consenting subjects will be asked to rinse with water and saliva allowed to accumulate in the floor of the mouth for approximately 2-3 minutes, after which they can spit in a sterile uricol container. A total of 12 samples were collected (6 from each clinic; for expected variances in oral health status) and immediately transferred to our microbiology laboratory for analysis (as described above), using the saliva as the media. All aforementioned experiments will be conducted in triplicate, for each concentration, single and combined (cock-tail), in simulated and human saliva. Statistical Analysis: one-way analysis of variance (ANOVA) for significant differences in MIC and MBC, followed by Duncan multiple range test for pair-wise comparisons will be performed using Statistical Package for Social Sciences, v.16, IBM Statistics (significance set at the 95% confidence level (p<0.05)). Dose-Response Curve: Given that agents are employed in concentrations internationally-approved for individual/single application, a dose-response curve (over the whole concentration range) for the combinatorial strategy is necessary. Hence, the aforementioned experiments include the determination of other inhibition concentration (IC) parameters: ICmin (lowest concentration leading to growth inhibition), IC50 (concentration that gives 50% growth inhibition), ICmax (minimum inhibition concentration and minimum bactericidal concentration), ICF (inhibition concentration factor) and Hill coefficient-related AS (activity slope). Diameter of inhibition zones (mm) determines antimicrobial activity. Statistical Analysis: ANOVA for inter-group differences by SPSS v.16, with (p<0.05).

### Example 1.3. Evaluation of the Immuno-Modulatory Properties of Single and Combined Synergistic Agents Against Cariogenic and Periodontal Disease Pathogens

In vitro evaluation of the immuno-modulatory potential of the dental floss of the invention is performed by isolation and culture of peripheral blood mononuclear cells (PBMCs) (collected from fresh blood samples of subjects attending the Clínica Odontológica (private)-Clínica Universidad de Los Andes (Las Condes, Santiago) and the Clínica Odontológica (public)-Centro de Salud de la Universidad de Los Andes (San Bernardo, Santiago) in 6-well plates for 7 days at 37° C. and 5% CO2. Cells are then seeded at 4×106/mL in 24-well plates (0.5 mL/well) and allowed to adhere for 3 days at 37° C. and 5% CO2. Non-adherent cells are removed by washing three times with pre-warmed (37° C.) PBS. Monocytes (which adhered to the plastic of the well) are then stimulated with different nanocapsules (Copper, Chlorhexidine and/or Fluoride; 1 ug/mL, 10 ug/mL and 100 ug/mL) for 24, 48 and 72 hrs. Cell viability is assessed by means of an alamarBLue assay: After stimulation, cell supernatant are removed and stored (−20° C.) for later cytokine analysis. New medium with alamarBlue reagent (catalog number DAL1100; Thermo Fisher Scientific, Waltham, Mass., USA) will be added directly to the wells. The cells are further incubated for 12 hours after which analysis of media fluorescence are performed at an excitation wavelength of 531 nm and emission of 590 nm using a plate reader (Victor3 1420 Multilabel Counter, PerkinElmer™). Final viability is calculated as % viability compared to control medium. The levels of pro-inflammatory cytokines TNF-a, IL-6 and IL-1B in the monocyte supernatants are detected using enzyme-linked immunosorbent assays (ELISA, R&D systems, Inc. MN, USA).

### Example 1.4. Incorporation of the Natural-Polymer Based Nanoformulations onto the Dental Floss

Dental flosses coated with prepared nano-formulations will be produced by first placing 40 mL of nano-particle (np) dispersions into 50 mL tubes. At least two commercially available mono- and multi-filament dental flosses such as (SupaGRIP by Piksters® and Mini-Flosser by TePe®) will be tested in four different groups (3 gadgets/each): (a) Distilled Water (control), (b) Copper np, (c) CHX-np and (d) F-np. Flosses will be immersed (secured by the holder) into the different nano-capsule solutions and stirred at 150 rpm for 24 hours following dry at room-temperature for another 24 hrs. During the above procedure, the nano-capsule formulations should coat the filaments of the Dental Floss. Dental flosses will be then stored at a room temperature/humidity until analysis. Will be repeated for bioFLOSS incorporating a, b & c.

### Example 1.5. Evaluation of the Total Nanocapsule Loading onto the Dental Floss and Release Kinetics

Briefly, the loading efficiency and drug release kinetics of the coated dental flosses are quantified by HPLC at different times in distilled water. Both nanocapsule morphology and distribution within the dental floss surface are determined using transmission electron microscopy.

### Example 2. Results

### Example 2.1. Characterization of NpCu

NpCu obtained from Otto Suhner Chile S.A and the NpCu with polymeric membrane synthesized with the technique layer-by-layer were characterized by measuring two main parameters: size and potential Z measured, using Nanosight equipment (NanoSight NS300, Malvern Panalytical Ltd, Grovewood Rd, Malvern WR14 1XZ, UK) suitable characterizing nanoparticles from 10 nm to 2000 nm in solution using NTA or Nanoparticle Tracking Analysis. In addition, the NpCu without polymeric membrane were characterized by SEM (Scanning Electron Microscope). The NpCu without polymeric membrane are characterized by an average size of 75.3±21.9 and a potential Z of −13.5±3.26. The NpCu with chitosan polymeric membrane (obtained by L-b-L Coating) have an average size of 112.7±6.7 and potential Z of +29.4±3.60, thus showing an improved stability. Said parameters can be adapted/controlled by modulating the characteristics of the polymeric membrane.

TABLE 1
Physical-chemical and rheological characterization
of NpCu and NpCu covered with chitosan

| | Average size (nm) | Potential Z (mV) |
|---|---|---|
| NpCu | 75.3 ± 21.9 | −13.5 ± 3.26 + |
| | | 14.5 ± 3.07 |
| NpCu with chitosan | 112.7 ± 6.7 | +29.4 + 3.60 |
| polymeric membrane | | |

### Example 2.2. Characterization of the Adhesion of NpCu with Polymeric Membrane to the Dental Floss

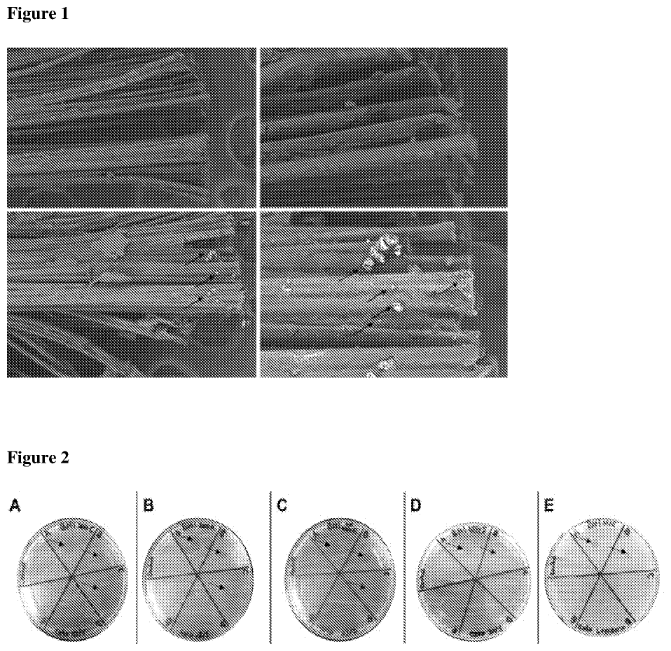

Dental floss with multi- or mono filaments was successfully embedded in a solution comprising NpCu with polymeric membrane, causing the incorporation of the NpCu in the surface of the dental floss. Through the analysis of the dental floss by SEM it was possible to observe the incorporation of the NpCu on the surface of the dental floss (see FIG. 1).

### Example 2.3. Determination of the Minimum Inhibitory Concentration (MIC) of NpCu

For the determination of the MIC, the antimicrobial activity of the NpCu was measured in different bacteria strains, through the observation of the formation of halos of inhibition in bacterial cultures: Streptococcus mutans serotype C, Streptococcus mutans serotype K, Streptococcus mutans serotype E, Streptococcus mutans serotype C (ATCC 25175), Staphylococcus epidermidis. A drop of NpCu was added at different concentrations, the bacterial strains were cultured in optimal conditions for 24 hours and the formation of halos of inhibition was observed (see FIG. 2 and Table 2).

TABLE 2

| | | | Antibacterial activity | | | | |
|---|---|---|---|---|---|---|---|
| | | | Streptococcus mutans | | | | |
| | | | Sero | Sero | Sero | | Staphylococcus |
| [NPCu] | | | C | K | E | 25175 | epidermidis |
| A: | 10 | mg/ml | (+) | (+) | (+) | (+) | (+) |
| B: | 5 | mg/ml | (+) | (+) | (+) | (+) | (+) |
| C: | 1 | mg/ml | (+/−) | (+/−) | (+/−) | (−) | (+/−) |
| D: | 0.1 | mg/ml | (−) | (−) | (−) | (−) | (−) |
| E: | 0.01 | mg/ml | (−) | (−) | (−) | (−) | (−) |

| | | | | | |
|---|---|---|---|---|---|
| Control: PBS 1x | (−) | (−) | (−) | (−) | (−) |

(+): Complete inhibition. (+/−): Partial inhibition. (−): without inhibition.

By observing the inhibition halos, it was possible to determine that the MIC is between 0.1 mg/ml and 5 mg/ml. After limiting the range in which there is inhibition of the microbial activity, bacterial growth inhibition trials were carried out in Staphylococcus epidermidis. Thus, Staphylococcus epidermidis was cultivated in liquid medium, different concentrations of NpCu between 0.1 and 5 mg/ml were added and incubated over night at 37° C., and the growth of bacteria in the different tubes was observed and summarized in Tables 3 to 8.

TABLE 3

| | | | Antibacterial activity |
|---|---|---|---|
| [NPCu] | | | Staphylococcus epidermidis |
| 1) | 5 | mg/ml | (+) |
| 2) | 4 | mg/ml | (+) |
| 3) | 3 | mg/ml | (+) |
| 4) | 2 | mg/ml | (+) |
| 5) | 1 | mg/ml | (−) |
| 6) | 0.8 | mg/ml | (−) |
| 7) | 0.5 | mg/ml | (−) |
| 8) | 0.3 | mg/ml | (−) |
| 9) | 0.1 | mg/ml | (−) |

| | |
|---|---|
| White | (−) |
| Control | (+) |

(+): Complete inhibition. (+/−): Partial inhibition. (−): without inhibition.

TABLE 4

| | | | Antibacterial activity |
|---|---|---|---|
| [NPCu] | | | Staphylococcus epidermidis |
| 1) | 2 | mg/ml | (+) |
| 2) | 1.9 | mg/ml | (+) |
| 3) | 1.8 | mg/ml | (+) |
| 4) | 1.7 | mg/ml | (+) |
| 5) | 1.6 | mg/ml | (+) |
| 6) | 1.5 | mg/ml | (+) |
| 7) | 1.4 | mg/ml | (+/−) |
| 8) | 1.3 | mg/ml | (+/−) |
| 9) | 1.2 | mg/ml | (+/−) |
| 10) | 1.1 | mg/ml | (−) |
| 11) | 1 | mg/ml | (−) |

| | |
|---|---|
| White | (−) |
| Control | (+) |

(+): Complete inhibition. (+/−): Partial inhibition. (−): without inhibition.

TABLE 5

| | | | Antibacterial activity |
|---|---|---|---|
| [NPCu] | | | Staphylococcus epidermidis |
| 1) | 1.8 | mg/ml | (+) |
| 2) | 1.7 | mg/ml | (+/−) |
| 3) | 1.6 | mg/ml | (+) |
| 4) | 1.5 | mg/ml | (+) |
| 5) | 1.4 | mg/ml | (−) |
| 6) | 1.3 | mg/ml | (+/−) |
| 7) | 1.2 | mg/ml | (+/−) |
| 8) | 1.1 | mg/ml | (−) |

| | |
|---|---|
| White | (−) |
| Control | (+) |

(+): Complete inhibition. (+/−): Partial inhibition. (−): without inhibition.

TABLE 6

| | | | Antibacterial activity |
|---|---|---|---|
| [NPCu] | | | Staphylococcus epidermidis |
| 1) | 2 | mg/ml | (−) |
| 2) | 1.9 | mg/ml | (+) |
| 3) | 1.8 | mg/ml | (+) |
| 4) | 1.7 | mg/ml | (+/−) |
| 5) | 1.6 | mg/ml | (−) |
| 6) | 1.5 | mg/ml | (−) |
| 7) | 1.4 | mg/ml | (−) |
| 8) | 1.3 | mg/ml | (−) |
| 9) | 1.2 | mg/ml | (−) |
| 10) | 1.1 | mg/ml | (−) |
| 11) | 1 | mg/ml | (−) |
| 12) | 0.5 | mg/ml | (−) |

| | |
|---|---|
| White | (−) |
| Control | (+) |

(+): Complete inhibition. (+/−): Partial inhibition. (−): without inhibition.

TABLE 7

| | | | Antibacterial activity |
|---|---|---|---|
| [NPCu] | | | Staphylococcus epidermidis |
| 1) | 2 | mg/ml | (+) |
| 2) | 1.9 | mg/ml | (−) |
| 3) | 1.8 | mg/ml | (−) |
| 4) | 1.7 | mg/ml | (−) |
| 5) | 1.6 | mg/ml | (−) |
| 6) | 1.5 | mg/ml | (−) |
| 7) | 1.4 | mg/ml | (−) |
| 8) | 1.3 | mg/ml | (−) |
| 9) | 1.2 | mg/ml | (−) |
| 10) | 1.1 | mg/ml | (−) |
| 11) | 1 | mg/ml | (−) |
| 12) | 0.5 | mg/ml | (−) |

| | |
|---|---|
| White | (−) |
| Control | (+) |

(+): Complete inhibition. (+/−): Partial inhibition. (−): without inhibition.

TABLE 8

| | | Antibacterial activity | | | | |
|---|---|---|---|---|---|---|
| [NPCu] | | Assay 1 | Assay 2 | Assay 3 | Assay 4 | Assay 5 |
| 5 | mg/ml | (+) | — | — | — | — |
| 4 | mg/ml | (+) | — | — | — | — |
| 3 | mg/ml | (+) | — | — | — | — |
| 2 | mg/ml | (+) | (+) | — | (−) | (+) |
| 1.9 | mg/ml | — | (+) | — | (+) | (−) |
| 1.8 | mg/ml | — | (+) | (+) | (+) | (−) |
| 1.7 | mg/ml | — | (+) | (+/−) | (+/−) | (−) |
| 1.6 | mg/ml | — | (+) | (+) | (−) | (−) |
| 1.5 | mg/ml | — | (+) | (+) | (−) | (−) |
| 1.4 | mg/ml | — | (+/−) | (−) | (−) | (−) |
| 1.3 | mg/ml | — | (+/−) | (+/−) | (−) | (−) |
| 1.2 | mg/ml | — | (+/−) | (+/−) | (−) | (−) |
| 1.1 | mg/ml | — | (−) | (−) | (−) | (−) |
| 1 | mg/ml | (−) | (−) | — | (−) | (−) |
| 0.8 | mg/ml | (−) | — | — | — | — |
| 0.5 | mg/ml | (−) | — | — | (−) | (−) |
| 0.3 | mg(ml | (−) | — | — | — | — |
| 0.1 | mg/ml | (−) | — | — | — | — |

| | | | | | |
|---|---|---|---|---|---|
| White | (−) | (−) | (−) | (−) | (−) |
| Control | (+) | (+) | (+) | (+) | (+) |

(+): Complete inhibition. (+/−): Partial inhibition. (−): without inhibition.

After co-incubating bacteria of the Staphylococcus epidermidis strain with NpCu solutions at different concentrations, it was possible to determine that the MIC of the NpCu for this bacterial type. It is possible to observe that there is a complete inhibition over 1.8 mg/ml in 3 out of the 4 trials. Between 1.7 mg/ml and 1.2 mg/ml it is possible to observe a partial inhibition of the microbial activity and under 1 mg/ml no inhibition is observed.

### Example 2.4. Synthesis and Physicochemical Characterization of Nanoparticles with Antimicrobials and Polymeric Cover

Copper nanoparticles were coated with natural polymers through the layer-by-layer self-assembly technique developed at University of Los Andes and were characterized by different parameters, then incorporated into dental flosses with different methodologies, the coated silk is also characterized.

2.4.1. Size Measurement.

Departing from copper nanoparticles (NPCu), nanoparticles with covers of different polymers were formulated through the layer-by-layer protocol, which were characterized before and after the coating to determine the changes in some of their physical parameters after the incorporation of polymers.

Copper Nanoparticles with Polymeric Chitosan Cover:

First, copper nanoparticles coated with chitosan polymer were formulated. They were characterized by measuring the size and potential Z parameters.

TABLE 9
Characterization of NpCu and NpCu covered with chitosan

| | Size (nm) | Potential Z (mV) |
|---|---|---|
| NpCu | 75.3 ± 21.9 | −13.5 ± 3.26 + |
| | | 14.5 ± 3.07 |
| NpCu covered with chitosan | 112.7 ± 6.7 | +29.4 ± 3.60 |

Copper Nanoparticles with Polymeric Alginate and Chitosan Cover:

Subsequently, copper nanoparticles were coated with alternating layers of the alginate and chitosan polymers, until obtaining nanoparticles with 6 layers (NP (AlCh) 3) (3 alginate and 3 chitosan) and 10 layers (NP (AlCh) 5) (5 of alginate and 5 of chitosan). These nanoparticles were characterized through size measurement in Nanosight NS300 equipment.

TABLE 10
Characterization of NpCu and NpCu (AlCh) 3 and NPCu (AlCh) 5

| | Production method | Size (nm) |
|---|---|---|
| NpCu | Collaboration with Nanotec Chile | 233.4 ± 9.7 nm |
| NpCu (AlCh)₃ | Layer-by-layer method | 73.5 ± 36.8 nm |
| NpCu (AlCh)₅ | Layer-by-layer method | 629.9 ± 288.4 nm |

The results obtained show that the size of the initial NpCu is very heterogeneous, which leads to polymeric nanoparticles of very variable sizes, both being coated with 3 and 5 bilayers, which would not allow observations representative of the changes in the size of the nanoparticles when coated with the polymers. Therefore, we sought to obtain more heterogeneous samples of the copper nanoparticles. A new formulation of NPCu, which was diluted in MiliQ water at a concentration of 500 ppm and size was measured in Nanosight team. Additionally, this solution was filtered with a pore of 0.45 μm.

TABLE 11
Characterization of unfiltered NpCu and filtered NPCu

| | Mean Size (nm) |
|---|---|
| NpCu | 231.0 +/− 7.6 nm |
| Filtered NpCu | 113.6 +/− 7.3 nm |

The new formulation of NPCu contains nanoparticles of a more homogeneous size than those used in the first formulations. In addition, the filtering after the preparation of the solutions allows limiting the size range of the nanoparticles present in the sample, which in turn will allow obtaining copper nanoparticles with a homogeneous polymeric shell.

Once homogeneous nanoparticles were obtained, the coating was carried out through the layer-by-layer method developed in-house. For this purpose, 2 alternatives were tested: 1) NPCu 500 ppm solution in MiliQ water and 2) NPCu powder.

TABLE 12
Characterization of NpCu and NpCu
covered with alginate and chitosan

| | | | Potential |
|---|---|---|---|
| | Production method | Size (nm) | Z (mV) |
| NpCu | Collaboration with | 122.5 ± 26.1 | −8.56 |
| | Nanotec Chile | | |
| NpCu (AlCh)₃ | In-house layer by | 243.3 ± 28.1 | 15.13 |
| (Solution) - batch 1 | layer method | | |
| NpCu (AlCh)₃ | In-house layer by | 284.3 ± 45.8 | 16.86 |
| (Solution) - batch 2 | layer method | | |
| NpCu (AlCh)₅ | In-house layer by | 477.9 ± 34.9 | 13.7 |
| (Solution) - batch 1 | layer method | | |
| NpCu (AlCh)₅ | In-house layer by | 464.7 ± 24.6 | 17.46 |
| (Solution) - batch 2 | layer method | | |
| NpCu (AlCh)₅ | In-house layer by | 272.5 ± 32.3 | 20.73 |
| (powder) | layer method | | |

2.4.2. Electronic Microscopy.

Copper nanoparticles with and without polymeric cover were analysed through scanning electronic microscopy. It was observed that the coating technique/methodology was successful, as well as the beneficial effect of the polymeric coating deposited layer by layer around the Cu nuclei on the general uniform dispersion in solution (which is directly associated with stability, bioactivity and pharmacokinetics over time).

2.4.3. Determination of the Optimal Methodology for Incorporating Nanoparticles into Dental Floss.

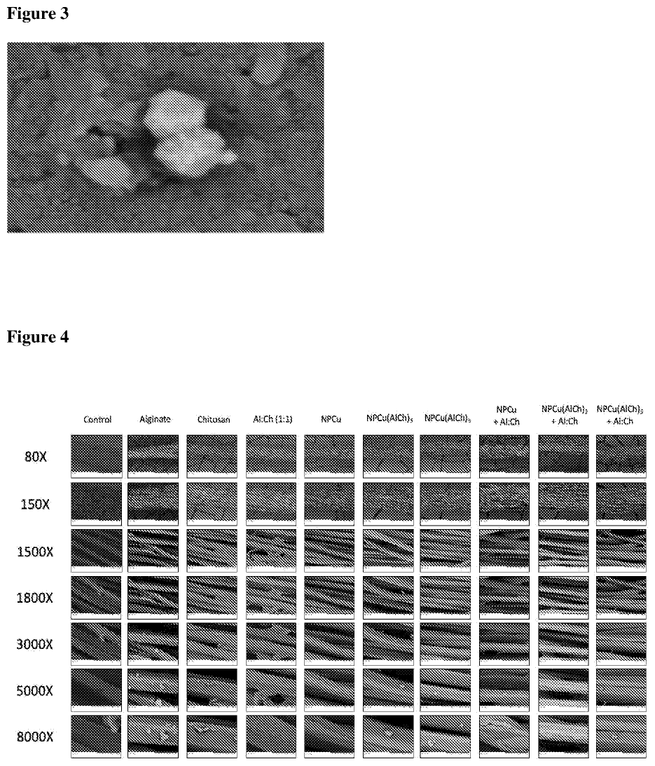

Multi and single filament dental floss (and FIG. 3) were embedded in polymeric coated NpCu solutions, for incorporation into dental floss. The flosses were observed by scanning electron microscopy (SEM).

Conclusion: Through the analysis of dental flosses by electron microscopy it was possible to observe the incorporation of NpCu. The formation of nanoparticle groups on dental floss was evidenced.

2.4.4. Commercial Dental Floss Analysis.

Once the proof of concept of the incorporation of the NPCu to dental floss was carried out, different dental flosses currently available in the market were analysed through scanning electron microscopy (SEM) to determine which of them is/are the most suitable for the incorporation of the NPCu.

After analyzing different types of commercial dental floss by electron microscopy, two specific samples were elected because their surface and fibers would make them more suitable for the incorporation of the NPCu on their surface. Both silks were treated with different mixtures of polymers and NPCu with and without polymeric cover as follows:
Alginate.
Chitosan.
Alginate: Chitosan (1:1)
NPCu.
NPCu (Al-Ch) 3.
NPCu (Al-Ch) 5.
Alginate: Chitosan (1:1)+NPCu.
NPCu (Al-Ch):3 Alginate: Chitosan (1:1)
NPCu (Al-Ch) 5+Alginate: Chitosan (1:1).

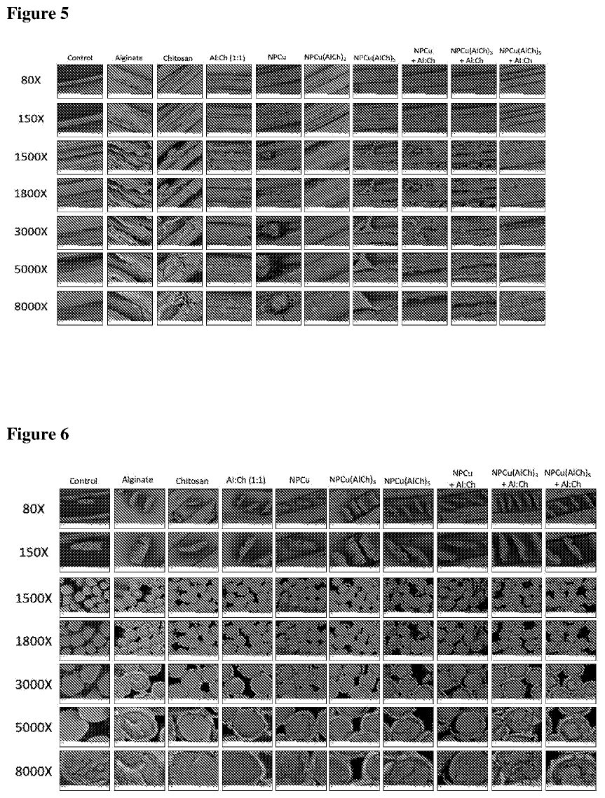

The treatments were applied in one or two cycles, depending on the type of coating that would be made to the silk, for 17 hours, at room temperature and moderate agitation followed by a drying cycle at room temperature for 6 hours. The samples that required more than one coating cycle were again treated with the same process. Subsequently, the samples were processed for observation by scanning electron microscopy (FIGS. 4 to 6).

2.4.5. Pharmacokinetic Characterization of Copper Nanoparticles Release.

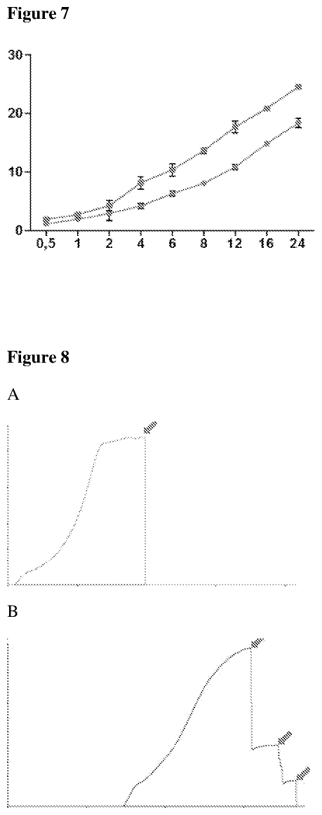

This pharmacokinetic characterization was made to determine the kinetic profiles of release of the NPCu from dental floss (18% vs. 24% in 24 min). See FIG. 7.

Conclusions: The first attempt to evaluate the pharmacokinetic profile of NpCu versus LpL coated with a single layer of chitosan, shows the potential effect and advantage of the multilayer polymer coating to control the release profile of NpCu (core) along the time, with the possibility of modulation of the release speed by changing the number of layers in the cover. Ongoing studies explore the pharmacokinetic release profiles of 3 (slow) and 5 (fast) NpCu samples, for 24 and 48 hours, using the most recent NpCu preparations.

2.4.6. Characterization of the Rheological Properties of the Dental Floss.

The dental floss tautness measurement was determined through the measurement in the TA.XT plus Texturometer. For this, first, the resistance of different untreated silks was determined. 10 cm pieces of three different samples of commercial silks were cut. Subsequently, the coating of one of the samples with different types of cover was performed according to the protocol described above, these correspond to (A) Alginate, (B) Chitosan, (C) Alginate:Chitosan (1:1), (D) NPCu 500 ppm and (E) NPCu (ALCM 3. The resistance of these samples was measured with the same configuration as for the control silks.

In general, two patterns of silk rupture are observed (FIG. 8), those that separate completely at the same time, and those in which the different fibers that make up the silks are broken at different times and strength. It is possible to identify in the number of breakpoints presented by the graph, with one for the first case and two or more for the second.

The following table (Table 13) shows the mechanical analysis of dental control flosses

TABLE 13
Mechanical analysis of dental control flosses. Number of break points
and average of the maximum strength and resistance (n = 15)

| | Number of | Maximum | Resistance |
|---|---|---|---|
| Sample | break points | strength (g) | (kg/mm) |
| Floss 3 | 1 | 2069.57 | 29.57 |
| Floss 4 | 1 | 4812.21 | 53.47 |
| Floss 5 | 1 | 3269.71 | 40.87 |

The mechanical properties, more specifically the resistance of dental flosses with different polymeric covers, were analysed. The results show that after treatment the tendency is to increase the number of breakpoints (Table 14), which indicates that the silk separates into different fibres and the application of forces of different magnitude is required to break them. The highest resistance was shown by the untreated silk (non-treated with polymers) while the lowest resistance was shown by the silk with polymeric nanoparticles on its surface.

Mechanical analysis of treated dental flosses. Number of break points
and average of the maximum strength and resistance (n = 3)

| | Number of | Maximum | Resistance |
|---|---|---|---|
| Sample | break points | strength | (kg/mm) |
| Floss 3 + Alginate | 2 | 1909.17 | 21.21 |
| Floss 3 + Chitosan | 1 | 2044.08 | 22.71 |
| Floss 3 + Alginate:Chitosan (1:1) | 2 | 1934.08 | 21.49 |
| Floss 3 + NPCu 500 ppm | 2 | 2251.92 | 28.15 |
| Floss 3 + NPCu (AlCh)₃ | 2 | 2074.68 | 17.45 |

Conclusions: After measuring the resistance of the silks, before and after being coated with different formulations, it is possible to observe that the incorporation of polymers and polymeric nanoparticles gives the silk the property of separating in its different fibres before breaking, this would be beneficial at the time of application since instead of being compact the silk would expand and it would be possible to cover a larger area in the interdental space.

### Example 3. Determination of the MIC Value of Bacteria Associated with Dental Plaque

3.1. Determination of MIC by Observation.

MIC is the lowest concentration of an antibacterial agent necessary to inhibit visible growth. For the determination the NpCu MIC, the growth inhibition of the different microorganisms was first measured when treated with the nanoparticles by observing the formation of inhibition halos in cultures solid media. For this, the microorganisms were grown and a drop of NpCu was added at different concentrations. Microorganisms were grown under optimal conditions for 24 hours and the formation of inhibition halos was observed.

TABLE 15
Observation of inhibition halos for determination of MIC.

| | |
|---|---|
| [NPCu] | Antimicrobial activity |

| | | | | | |
|---|---|---|---|---|---|
| (ppm) | M1 | M2 | M3 | M4 | M5 |

| | | | | | | |
|---|---|---|---|---|---|---|
| A: | 10.000 | (+) | (+) | (+) | (+) | (+) |
| B: | 5.000 | (+) | (+) | (+) | (+) | (+) |
| C: | 1.000 | (+/−) | (+/−) | (+/−) | (−) | (+/−) |
| D: | 100 | (−) | (−) | (−) | (−) | (−) |
| E: | 10 | (−) | (−) | (−) | (−) | (−) |

| | | | | | |
|---|---|---|---|---|---|
| Control: PBS 1x | (−) | (−) | (−) | (−) | (−) |

(+): Complete inhibition. (+/−): Partial inhibition. (−): Without inhibition.

Through the observation of the inhibition halos formed, it was possible to determine that the minimum inhibitory concentration is between 100 and 5000 ppm.

Once the range in which there is inhibition was stablished, bacterial growth inhibition assays in microorganism 5 (M5) were performed in order to determine the value of the minimum inhibitory concentration. For this, M5 was cultured in liquid medium and different concentrations of NpCu were added and incubated over night at 37° C. It was observed whether there was growth of bacteria in the different tubes.

TABLE 16
Observations made for antimicrobial activity in M5.

| [NPCu] | |
|---|---|
| (ppm) | Antimicrobial activity |

| | | |
|---|---|---|
| 1) | 5.000 | (+) |
| 2) | 4.000 | (+) |
| 3) | 3.000 | (+) |
| 4) | 2.000 | (+) |
| 5) | 1.000 | (−) |
| 6) | 800 | (−) |
| 7) | 500 | (−) |
| 8) | 300 | (−) |
| 9) | 100 | (−) |

| | |
|---|---|
| White | (−) |
| Control | (+) |

(+): Complete inhibition. (+/−): Partial inhibition. (−): Without inhibition.

TABLE 17
Observations made for antimicrobial activity in M5.

| [NPCu] | |
|---|---|
| (ppm) | Antimicrobial activity |

| | | |
|---|---|---|
| 1) | 2.000 | (+) |
| 2) | 1.900 | (+) |
| 3) | 1.800 | (+) |
| 4) | 1.700 | (+) |
| 5) | 1.600 | (+) |
| 6) | 1.500 | (+) |
| 7) | 1.400 | (+) |
| 8) | 1.300 | (+/−) |
| 9) | 1.200 | (+/−) |
| 10) | 1.100 | (−) |
| 11) | 1.000 | (−) |

| | |
|---|---|
| White | (−) |
| Control | (+) |

(+): Complete inhibition. (+/−): Partial inhibition. (−): Without inhibition.

TABLE 18
Observations made for antimicrobial activity in M5.

| [NPCu] | |
|---|---|
| (ppm) | Antimicrobial activity |

| | | |
|---|---|---|
| 1) | 1.800 | (+) |
| 2) | 1.700 | (+/−) |
| 3) | 1.600 | (+) |
| 4) | 1.500 | (+) |
| 5) | 1.400 | (−) |
| 6) | 1.300 | (+/−) |
| 7) | 1.200 | (+/−) |
| 8) | 1.100 | (−) |

| | |
|---|---|
| White | (−) |
| Control | (+) |

(+): Complete inhibition. (+/−): Partial inhibition. (−): Without inhibition.

TABLE 19
Observations made for antimicrobial activity in M5.

| [NPCu] | |
|---|---|
| (ppm) | Antimicrobial activity |

| | | |
|---|---|---|
| 1) | 2.000 | (−) |
| 2) | 1.900 | (+) |
| 3) | 1.800 | (+) |
| 4) | 1.700 | (+/−) |
| 5) | 1.600 | (−) |
| 6) | 1.500 | (−) |
| 7) | 1.400 | (−) |
| 8) | 1.300 | (−) |
| 9) | 1.200 | (−) |
| 10) | 1.100 | (−) |
| 11) | 1.000 | (−) |
| 12) | 500 | (−) |

| | |
|---|---|
| White | (−) |
| Control | (+) |

(+): Complete inhibition. (+/−): Partial inhibition. (−): Without inhibition.

TABLE 20
Observations made for antimicrobial activity in M5.

| [NPCu] | |
|---|---|
| (ppm) | Antimicrobial activity |

| | | |
|---|---|---|
| 1) | 2.000 | (+) |
| 2) | 1.900 | (−) |
| 3) | 1.800 | (−) |
| 4) | 1.700 | (−) |
| 5) | 1.600 | (−) |
| 6) | 1.500 | (−) |
| 7) | 1.400 | (−) |
| 8) | 1.300 | (−) |
| 9) | 1.200 | (−) |
| 10) | 1.100 | (−) |
| 11) | 1.000 | (−) |
| 12) | 500 | (−) |

| | |
|---|---|
| White | (−) |
| Control | (+) |

(+): Complete inhibition. (+/−): Partial inhibition. (−): Without inhibition.

TABLE 21
Summary observations made for antimicrobial activity in M5.

| | |
|---|---|
| [NPCu] | Antimicrobial activity |

| (ppm) | Assay 1 | Assay 2 | Assay 3 | Assay 4 | Assay 5 |
|---|---|---|---|---|---|
| 5.000 | (+) | — | — | — | — |
| 4.000 | (+) | — | — | — | — |
| 3.000 | (+) | — | — | — | — |
| 2.000 | (+) | (+) | — | (−) | (+) |
| 1.900 | — | (+) | — | (+) | (−) |
| 1.800 | — | (+) | (+) | (+) | (−) |
| 1.700 | — | (+) | (+/−) | (+/−) | (−) |
| 1.600 | — | (+) | (+) | (−) | (−) |
| 1.500 | — | (+) | (+) | (−) | (−) |
| 1.400 | — | (+/−) | (−) | (−) | (−) |
| 1.300 | — | (+/−) | (+/−) | (−) | (−) |
| 1.200 | — | (+/−) | (+/−) | (−) | (−) |
| 1.100 | — | (−) | (−) | (−) | (−) |
| 1.000 | (−) | (−) | — | (−) | (−) |
| 800 | (−) | — | — | — | — |
| 500 | (−) | — | — | (−) | (−) |
| 300 | (−) | — | — | — | — |
| 100 | (−) | — | — | — | — |
| White | (−) | (−) | (−) | (−) | (−) |
| Control | (+) | (+) | (+) | (+) | (+) |

(+): Complete inhibition. (+/−): Partial inhibition. (−): Without inhibition.

Conclusions: After incubating the microorganisms with NpCu solutions at different concentrations, it was possible to determine that the minimum inhibitory concentration of NpCu for M5 was 1,800 ppm in 3 out of 4 trials. Between 1700 and 1200 ppm it is possible to observe a partial inhibition of microbial activity, and below 1000 ppm no inhibition is observed.

3.2. Determination of MIC by Absorbance Measurement.

In order to determine the MIC of the non-coated copper nanoparticles (CuNp), 96-well plates were prepared with decreasing concentrations of NPCu, which were modified for the different bacteria according to the results obtained in the process, with the purpose of finding the WCC. Then the diluted inoculum was added. Plates were incubated according to bacterial conditions and measured daily on the spectrophotometer at 620 nm.

L. Casei:

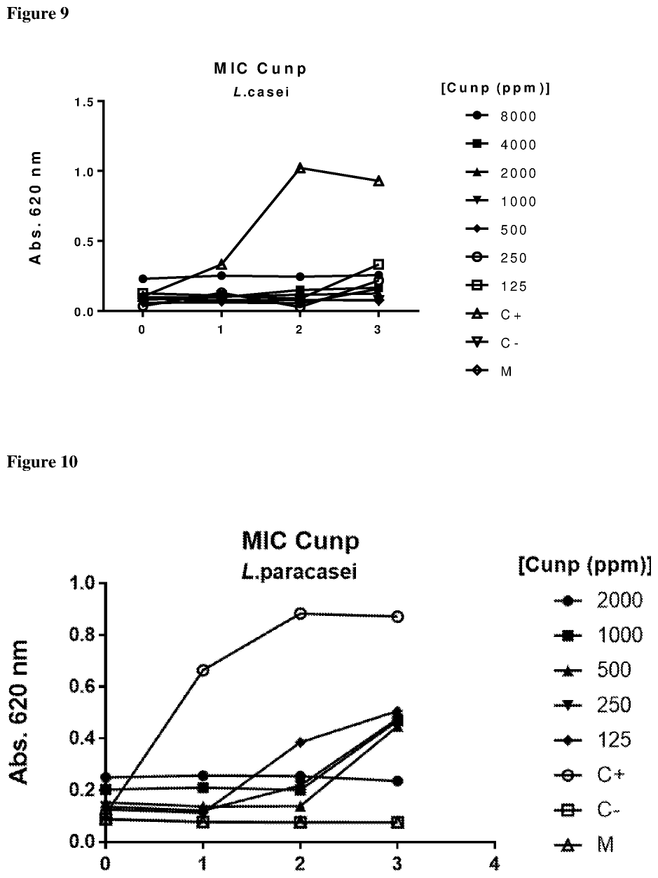

MIC was determined for NPCu in L. Casei at concentrations between 125 and 8000 ppm. The results obtained indicate that the minimum inhibitory concentration is 125 ppm (FIG. 9).

L. Paracasei:

The MIC for NPCu in L. Paracasei was determined at concentrations between 125 and 2000 ppm. The results obtained indicate that the minimum inhibitory concentration is 2000 ppm for this bacterium (FIG. 10).

S. mutans:

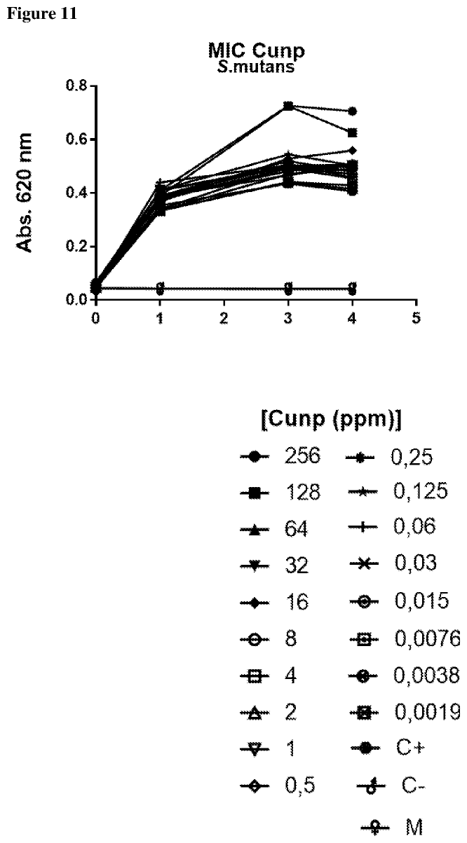

The MIC for NPCu in S. mutans was determined at concentrations between 256 and 0.0019 ppm. The results obtained indicate that the minimum inhibitory concentration is around 256 ppm (FIG. 11).

Conclusions: After incubating the microorganisms with NpCu solutions at different concentrations, it was possible to determine that the minimum inhibitory concentration of the NpCu is 2000, 256 and 125 ppm. In this sense, the MIC of L. paracasei is similar to that obtained by observation in previous trials. The importance of developing this methodology is the reduction of the impact of NPCu precipitation and thus it is possible to measure absorbance, obtaining a much more sensitive method that allows to observe inhibition at lower concentrations and smaller differences than through observation.

3.3. Biofilm Tests.

To obtain biofilms, 96-well plates with different concentrations of NPCu were prepared and the diluted inoculum was added at these concentrations. The plates were incubated for 48 hours and were revealed using violet crystal to determine the bacteria amount that formed biofilm according to each treatment.

L. Casei:

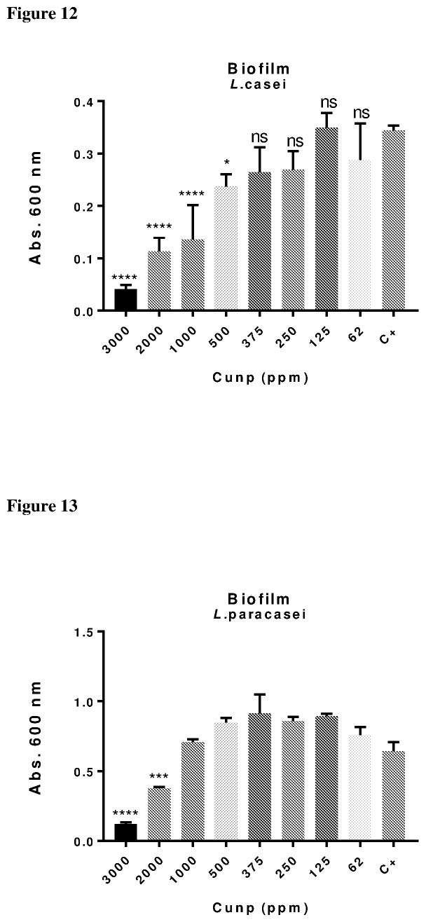

The ability to form biofilms in the presence of different NPCu concentrations in L. Casei, at concentrations between 62 and 3000 ppm, was determined. The results obtained indicate that the minimum concentration at which a significant decrease in biofilm formation is observed is 500 ppm (FIG. 12).

L. Paracasei:

The ability to form biofilms in the presence of different NPCu concentrations in L. Paracasei at concentrations between 62 and 3000 ppm was determined. The results obtained indicate that the minimum concentration at which a significant decrease in biofilm formation is observed is at 2000 ppm (FIG. 13).

Conclusions: The ability to form biofilms in the presence of NPCu was determined. It was observed that over 500 and 2000 ppm of NPCu, the bacteria are not able to form these structures. These values are similar to those obtained for some of the microorganisms for MIC, which will allow adjusting the administered dose.

3.4. Determination of the Antimicrobial Activity of Nanoparticles is a Mixed Biofilm Model.

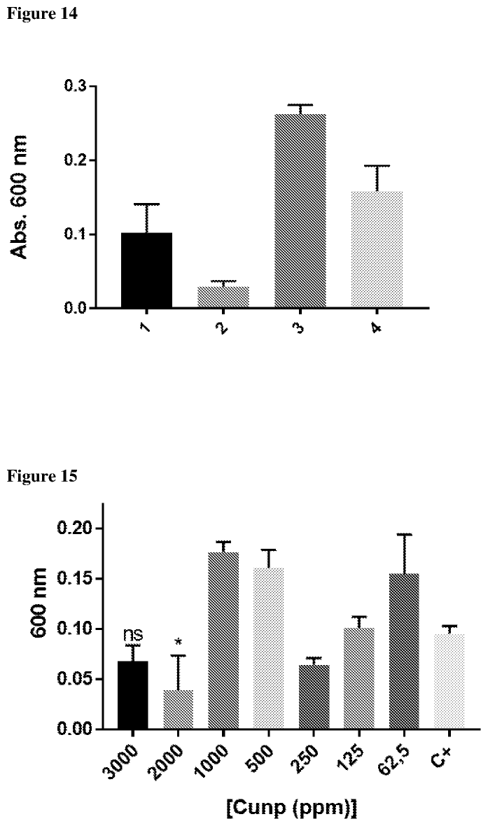

In the mixed biofilm model, a sample of human saliva is used as inoculum to obtain a closer reflection of what happens in the oral cavity. For this, healthy donors, without a history of oral pathologies, will remain without eating 2 hours before obtaining the sample and their last oral hygiene will be 12 hours before. At least 5 ml of unstimulated saliva were obtained in Falcon tubes and prepared 96-well plates with saliva from the 4 donors to determine the ability to generate biofilm of the samples. In addition, a 96-well plate was prepared with decreasing concentrations of NPCu (3000-62 ppm) mixed with saliva from randomly selected donors 1 and 2. The plates are incubated for 48 hours and revealed using violet crystal which allows to dye and fix the bacteria that form film to determine the amount of bacteria that are capable of forming biofilms, both in the samples alone and with treatment. See FIG. 14. It is observed that donor 3 is the one that generates a larger biofilm compared to the other samples. In any case, the bacterial load is less than what could be obtained from a saturated culture of bacteria.

According to FIG. 15, these preliminary results show that the first concentration of NPCu that shows a significant decrease in biofilm formation is at 2000 ppm, which is consistent with the MIC values and biofilm assays shown above.

3.5. Determination of the Cytotoxicity of Nanoparticles in Tooth Support Tissues.

In vitro assays were performed on different cell lines to determine those concentrations at which the nanoparticles could be cytotoxic to the cells. Cytotoxicity was determined through the measurement of cell viability using the PrestoBlue reagent. Two control conditions were added for each experiment: Positive control (ctrl +), which corresponds to the basal condition of the cells only with normal culture medium, and a negative control (ctrl −) in which the cells were incubated with methanol 70% for 30 minutes (this was used as a cell death control). All conditions in triplicate.

Cal27 Cells:

a) NPCu without Polymeric Cover:

For this cell type, 25,000 cells were seeded for each condition in 96-well plates, incubated with NPCu solutions of different concentrations for 24 hours. The measurement was then performed with PrestoBlue for 1 hour according to the manufacturer's instructions.

TABLE 22
Percentage of cell viability after incubation with NpCu in Cal27 cells

| [NPCu] | | | | | | | | | | |
|---|---|---|---|---|---|---|---|---|---|---|
| (ppm) | 100 | 750 | 500 | 250 | 100 | 75 | 50 | 10 | CTRL+ | CTRL− |
| % cell | −3.8 | −10.5 | −3.1 | −3.6 | 0.9 | 13.3 | 42.7 | 91.5 | 100.0 | −4.3 |
| viability | | | | | | | | | | |

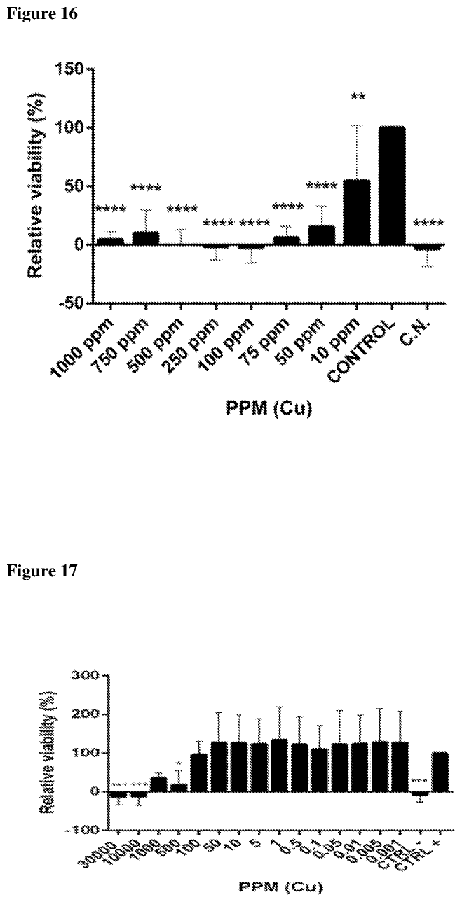

The results show a decrease in cell viability after incubation with NpCu at all concentrations applied. However, these results would not be representative of the effect of NpCu on cell viability since the Cal27 cells used were not in optimal conditions, presenting a low adhesion to the plaque so that the lower number of cells after incubation could due to a detachment of these and not to the direct cytotoxic effect of the NPCu. Therefore, other cell lines were tested, which were in optimal conditions to determine the cytotoxic effect of the NPCu by eliminating the adhesion factor that could alter the results obtained (see FIG. 16).

MC3T3-E1 Cells:

To perform the tests with this cell line, 2000 cells were seeded by treatment in 96-well plates, which were incubated for 24 h with NPCu solutions of different concentrations. Subsequently, cell viability was measured using PrestoBlue reagent (1 hour) according to the manufacturer's instructions.

a) NPCu without Polymeric Cover:

TABLE 23
Percentage of cell viability after incubation with NpCu in MC3T3-E1 cells

| [NPCu] | | | | | | | | | | | | | | | | | |
|---|---|---|---|---|---|---|---|---|---|---|---|---|---|---|---|---|---|
| (ppm) | 30000 | 10000 | 1000 | 500 | 100 | 50 | 10 | 5 | 1 | 0.5 | 0.1 | 0.05 | 0.01 | 0.005 | 0.001 | Ctrl− | Ctrl+ |
| % cell | −12.6 | −11.4 | 35.6 | 17.8 | 95.7 | 127.4 | 126.1 | 123.0 | 134.3 | 122.2 | 110.1 | 122.5 | 123.9 | 128.2 | 126.5 | −8.3 | 100.0 |
| viability | | | | | | | | | | | | | | | | | |

The data obtained show that there is a statistically significant decrease in cell viability when the nanoparticles are in concentrations greater than 500 ppm. See FIG. 17.

b) NPCu with Polymeric Cover:

Polymeric coated NPCu were used and cell viability was measured after incubation.

NPCu Solution (see FIG. 18):

TABLE 24
Percentage of cell viability after incubation with NpCu (AICh) 5 in MC3T3-E1 cells

| [NPCu(AlCh)₅] | | | | | | | | CTRL | | |
|---|---|---|---|---|---|---|---|---|---|---|
| (particles/mL) | 3.83E+007 | 3.83E+006 | 3.83E+005 | 3.83E+004 | 3.83E+003 | 3.83E+002 | 3.83E+001 | LAYERS | CTRL− | CTRL+ |
| % cell | 102.6 | 100.4 | 94.4 | 88.7 | 92.1 | 93.7 | 99.1 | 90.2 | 0.0 | 100.0 |
| viability | | | | | | | | | | |

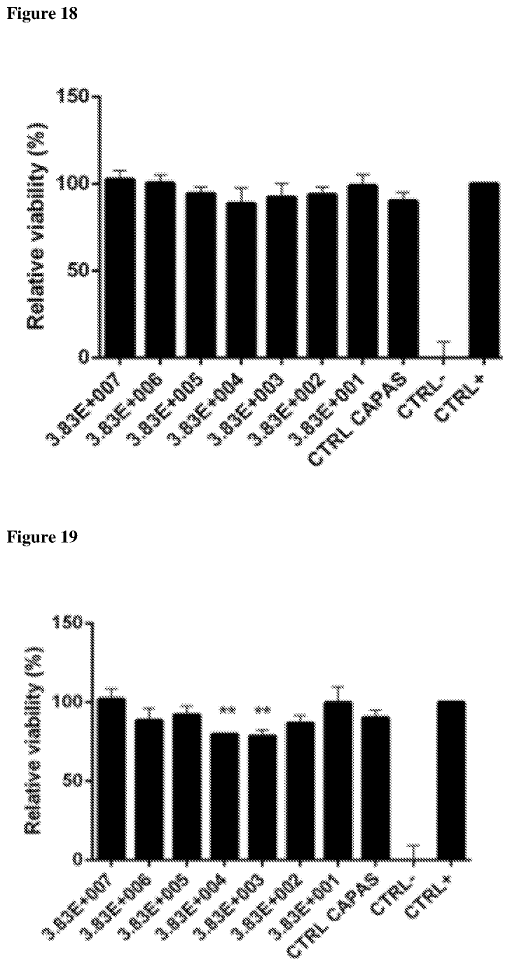

NPCu Powder (see FIG. 19):

TABLE 25
Percentage of cell viability after incubation with NpCu (AlCh) 5 from NPCu powder in MC3T3-E1 cells

| [NPCu(AlCh)₅] | | | | | | | | CTRL | | |
|---|---|---|---|---|---|---|---|---|---|---|
| (particles/mL) | 3.83E+007 | 3.83E+006 | 3.83E+005 | 3.83E+004 | 3.83E+003 | 3.83E+002 | 3.83E+001 | LAYERS | CTRL− | CTRL+ |
| % cell | 101.9 | 88.6 | 92.1 | 79.8 | 78.5 | 86.9 | 99.9 | 90.2 | 0.0 | 100.0 |
| viability | | | | | | | | | | |

SCC-9 Cells:

In this cell type, 2000 cells were seeded in 96-well plate, incubated with NPCu solutions of different concentrations for 24 hours. Then the measurement was performed with PrestoBlue, for 1 hour, according to the manufacturer's instructions.

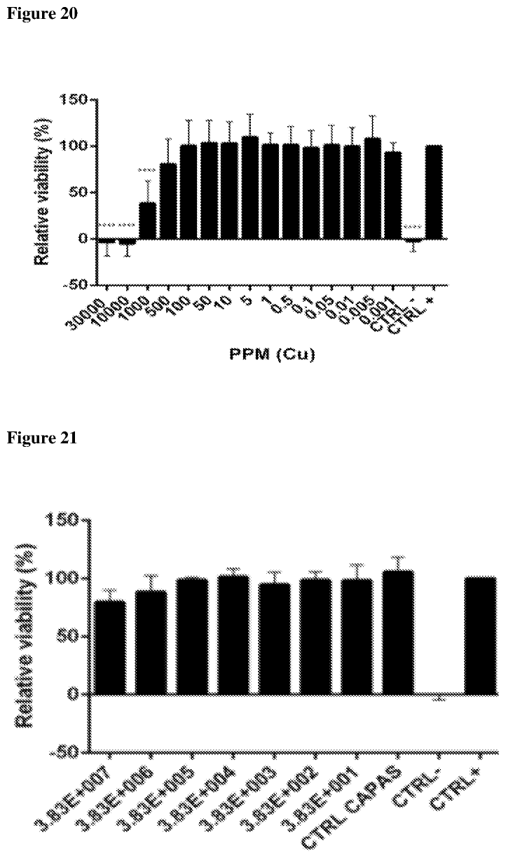

a) NPCu without Polymeric Cover (See FIG. 20):

TABLE 26
Percentage of cell viability after incubation with NpCu in SCC-9 cells

| [NPCu(AlCh)₅] | | | | | | | | | | | | | | | | | |
|---|---|---|---|---|---|---|---|---|---|---|---|---|---|---|---|---|---|
| (particles/mL) | 30000 | 10000 | 1000 | 500 | 100 | 50 | 10 | 5 | 1 | 0.5 | 0.1 | 0.05 | 0.01 | 0.005 | 0.001 | Ctrl− | Ctrl+ |
| % cell | −3.5 | −4.9 | 38.5 | 80.6 | 100.6 | 103.4 | 103.2 | 109.9 | 101.8 | 101.7 | 98.4 | 101.3 | 99.9 | 107.9 | 93.0 | −2.0 | 100.0 |
| viability | | | | | | | | | | | | | | | | | |

b) NPCu with Polymeric Cover:

Polymeric coated NPCu were used, cell viability was measured after incubation with 10 layers NPCu.

NPCu Solution (see FIG. 21):

TABLE 27
Percentage of cell viability after incubation with NpCu (AlCh) 5 in SCC-9 cells

| [NPCu(AlCh)₅] | | | | | | | | CTRL | | |
|---|---|---|---|---|---|---|---|---|---|---|
| (particles/mL) | 3.83E+007 | 3.83E+006 | 3.83E+005 | 3.83E+004 | 3.83E+003 | 3.83E+002 | 3.83E+011 | LAYERS | CTRL− | CTRL+ |
| % cell | 79.7 | 88.2 | 98.6 | 101.3 | 94.7 | 98.5 | 98.3 | 105.8 | 0.0 | 100.0 |
| viability | | | | | | | | | | |

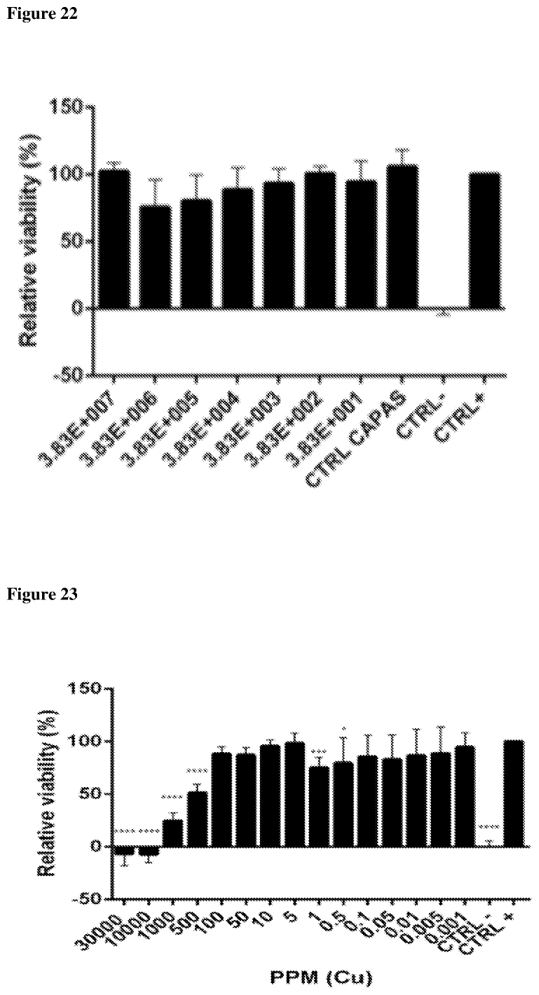

NPCu Powder (see FIG. 22):

TABLE 28
Percentage of cell viability after incubation with NpCu (AlCh) 5 from NPCu powder in SCC-9 cells

| [NPCu(AlCh)₅] | | | | | | | | CTRL | | |
|---|---|---|---|---|---|---|---|---|---|---|
| (particles/mL) | 3.83E+007 | 3.83E+006 | 3.83E+005 | 3.83E+004 | 3.83E+003 | 3.83E+002 | 3.83E+001 | LAYERS | CTRL− | CTRL+ |
| % cell | 102.3 | 75.6 | 80.2 | 88.7 | 93.2 | 100.7 | 94.4 | 105.8 | 0.0 | 100.0 |
| viability | | | | | | | | | | |

HDFn Cells:

In this cell type, 2000 cells were seeded in 96-well plate, incubated with NPCu solutions of different concentrations for 24 hours. Then the measurement was performed with PrestoBlue, for 1 hour, according to the manufacturer's instructions.

a) NPCu without Polymeric Cover (See FIG. 23):

TABLE 29
Percentage of cell viability after incubation with NpCu in HDFn cells

| [NPCu(AlCh)₅] | | | | | | | | | | | | | | | | | |
|---|---|---|---|---|---|---|---|---|---|---|---|---|---|---|---|---|---|
| (particles/mL) | 30000 | 10000 | 1000 | 500 | 100 | 50 | 10 | 5 | 1 | 0.5 | 0.1 | 0.05 | 0.01 | 0.005 | 0.001 | Ctrl− | Ctrl+ |
| % cell | −6.6 | −7.1 | 24.7 | 51.4 | 88.4 | 87.2 | 95.8 | 98.5 | 75.3 | 79.6 | 85.8 | 82.9 | 86.8 | 88.7 | 94.8 | 0.0 | 100.0 |
| viability | | | | | | | | | | | | | | | | | |

For the HDFn fibroblast cell line, NPCu have cytotoxicity above 500 ppm. They also show a significant decrease in cell viability when they are at concentrations of 1 ppm and 0.5 ppm.

b) NPCu with polymeric cover:

Polymeric coated NPCu were used, cell viability was measured after incubation with 10 layers NPCu.

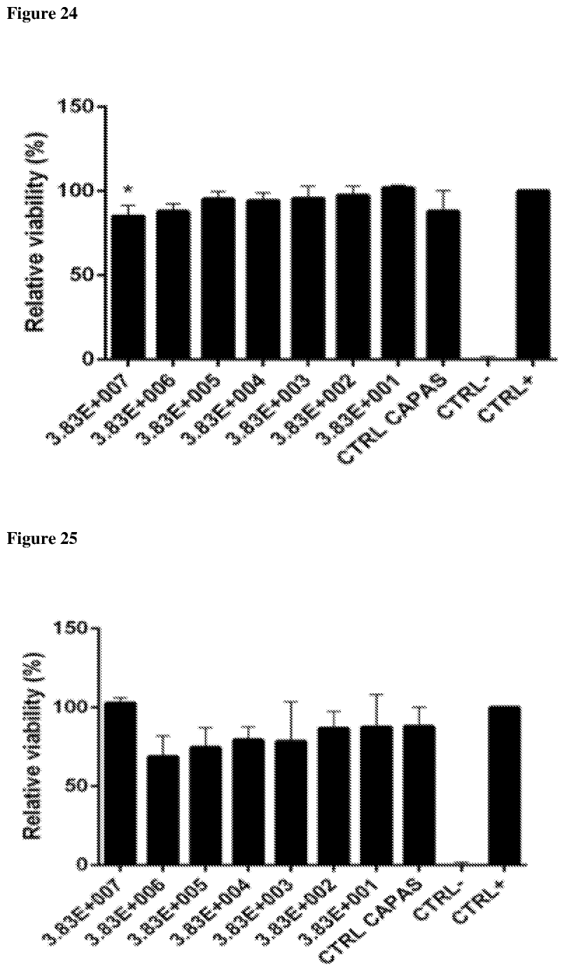

NPCu Solution (see FIG. 24):

TABLE 30
Percentage of cell viability after incubation with NpCu (AlCh) 5 in HDFn cells

| [NPCu(AlCh)₅] | | | | | | | | CTRL | | |
|---|---|---|---|---|---|---|---|---|---|---|
| (particles/mL) | 3.83E+007 | 3.83E+006 | 3.83E+005 | 3.83E+004 | 3.83E+003 | 3.83E+002 | 3.83E+001 | LAYERS | CTRL− | CTRL+ |
| % cell | 102.9 | 68.8 | 74.7 | 79.4 | 78.5 | 86.7 | 87.6 | 87.9 | 0.0 | 100.0 |
| viability | | | | | | | | | | |

NPCu Powder (see FIG. 25):

TABLE 31
Percentage of cell viability after incubation with NpCu (AlCh) 5 from NPCu powder at HDFn cells

| [NPCu(AlCh)₅] | | | | | | | | CTRL | | |
|---|---|---|---|---|---|---|---|---|---|---|
| (particles/mL) | 3.83E+007 | 3.83E+006 | 3.83E+005 | 3.83E+004 | 3.83E+003 | 3.83E+002 | 3.83E+001 | LAYERS | CTRL− | CTRL+ |
| % cell | 85.1 | 87.9 | 95.3 | 94.3 | 95.6 | 97.4 | 102.0 | 87.9 | 0.0 | 100.0 |
| viability | | | | | | | | | | |

Conclusion: Cytotoxicity was measured in 3 cell lines: MC3T3-E1, SCC-9 and HDFn, which correspond to osteoblastic cells, squamous cell carcinoma epithelial cells in human tongue and dermal fibroblasts respectively. The data obtained from the measurements made in these 3 cell types show that the copper nanoparticles have cytotoxicity over 500 or 1000 ppm depending on the cell type. On the other hand, it is possible to observe that by adding the polymeric coating to the nanoparticles, they only present cytotoxicity in 3 cases. This indicates that the polymeric shell functions as a structure that covers the metal nanoparticle and protects the cell from possible toxicity. In addition, the controls performed with the polymer mixture show that the polymeric shell would not be a cytotoxic agent for the cells.

1. Dental floss coated with a composition comprising metal nanoparticles wherein said nanoparticles have an antibacterial, antifungal and/or immunostimulating effect and the nanoparticles are further characterized by comprising a polymeric membrane made of at least one natural occurring polymer which causes the incorporation of the nanoparticles on the surface of the dental floss.

2. Dental floss, according to claim 1, wherein the nanoparticle is a copper, silver or lithium nanoparticle.

3. Dental floss, according to any of the claim 1 or 2, wherein the natural occurring polymer is selected from the list comprising: chitosan, gelatine, alginate, cellulose, hyaluronic acid, albumin, or any salts derived thereof.

4. Dental floss, according to any of the claims 1 to 3, coated with a composition comprising copper nanoparticles having a polymeric membrane comprising chitosan.

5. Use of metal nanoparticles which have an antibacterial, antifungal and/or immunostimulating effect and comprise a polymeric membrane made of at least one natural occurring polymer, for coating a dental floss.

6. Use, according to the claim 5, wherein the nanoparticle is a copper, silver or lithium nanoparticle.

7. Use, according to claims 5 to 6, wherein the natural occurring polymer is selected from the list comprising: chitosan, gelatine, alginate, cellulose, hyaluronic acid, albumin, or any salts derived thereof.

8. Use, according to claims 5 to 7, wherein the nanoparticles are copper nanoparticles having a polymeric membrane comprising chitosan.

9. Dental floss coated with a composition according to any of the claims 1 to 4 for use in the prevention or treatment of caries and/or periodontal disease.

10. Dental floss for use, according to claim 9, wherein the prevention or treatment of caries and/or periodontal disease is carried out by inhibiting the bacteria strains Streptococcus mutans serotype C, Streptococcus mutans serotype K, Streptococcus mutans serotype E, Streptococcus mutans serotype C (ATCC 25175) or Staphylococcus epidermidis, thereby inhibiting oro0dental biofilm formation, onset and/or progression of dental diseases such as dental decay/caries and gingival/periodontal recession.

11. Method for obtaining a dental floss according to any of the claims 1 to 4 comprising the steps:
a. Preparing a composition comprising metal nanoparticles wherein said nanoparticles have an antibacterial, antifungal and/or immunostimulating effect and the nanoparticles are further characterized by comprising a polymeric membrane made of at least one natural occurring polymer,
b. Step-wise adsorption of different polymer coating assembled layer-by-layer onto the floss thereby creating multi-compartments around it able to be loaded with therapeutic or preventive active ingredients, and
c. Drying the composition prepared according to the step a) on the dental floss.

12. Method, according to the claim 11, wherein the polymeric membrane is prepared by layer-by-layer self-assembly of the polymer blends.
